(19) United States
(12) Patent Application Publication
(10) Pub. No.: US 2005/0165409 A1
(43) Pub. Date: Jul. 28, 2005

(54) Removable implant fixing device

(76) Inventor: Rolf Eckmiller, Neuss (DE)

Correspondence Address: Robert W Becker & Associates, Suite B, 707 Highway 66 East, Tijeras, NM, 87059, US

(21) Appl. No.: 10/433,246

(22) PCT Filed: Dec. 3, 2001
(86) PCT No.: PCT/EP01/14077

(30) Foreign Application Priority Data
Dec. 1, 2000 (DE) 10060029.8

(51) Int. Cl. A61B 17/08

(52) U.S. Cl. 606/107

(57) ABSTRACT

The invention relates to a removable anchoring element (3) for an implantable microcontact foil (2) used as a neuroimplant, especially for retina implants. The anchoring head rises above the surface of the implant, for fixing purposes, and the fixing device can be removed during the re-explantation or anchoring process by pulling the implant foil (2) beneath the anchoring head.

The present invention relates to a fixing device for implantable microcontact foils as neuroimplants, in particular retina implants with the features of the preamble of claim 1.

Fixings of this kind are known, for example, from specification W000/40161. A description of a retina implant attached with the known fixing devices in the eye is described in the article “Development of flexible stimulation devices for a retina implant system” by Stieglitz et al. in Proceedings, 19th International Conference-IEEE/EMBS, Oct. 30-Nov. 2, 1997, Chicago, Ill., USA, page 2.307-2.310.

According to the state of the art, microcontact foils for neuroprostheses, in particular the said retina implants as vision prostheses, are attached epiretinal to the retina with micro tacks, where these micro tacks are pushed through holes in the microcontact foil and anchored through the retina, the pigmented epithelium and the choroid to the sclera. As a result, these fixings cannot be removed again without significant lesions in the retina, so that re-explantation of the microcontact foil is extremely difficult.

The object of the present invention is therefore to create a removable fixing or holding of the microcontact foils in particular to retina tissue.

This object is achieved with a fixing with the features of claim 1.

Because the anchoring head rises above or beyond the implant surface for the purpose of fixing and the fixing can be removed for re-explantation by pulling the implant foil beneath the anchoring head, the microcontact foil can be removed without necessarily damaging the tissue below. For example, a new microcontact foil can be fitted to replace a faulty microcontact foil or as a more powerful implant following technical improvements without fear of further damage to the tissue.

If sideways pulling of the microcontact foil below the anchoring heads is undesirable, the anchoring head can also be designed removable from a base body which remains in the tissue. It can further be provided that the anchoring head is mounted swivelling in the manner of a toggle closure and can be swivelled for holding over the top of the microcontact foil. To remove the microcontact foil in a direction perpendicular to the tissue surface, the head is swivelled so that the microcontact foil is released. Finally, for particular applications, it can also be provided that an anchoring structure protruding through the tissue is attached to the tissue and carries a removable fixing head so that this fixing head can be inserted and removed without damaging the tissue.

Two embodiments of the present invention are described below with reference to the drawings. These show:

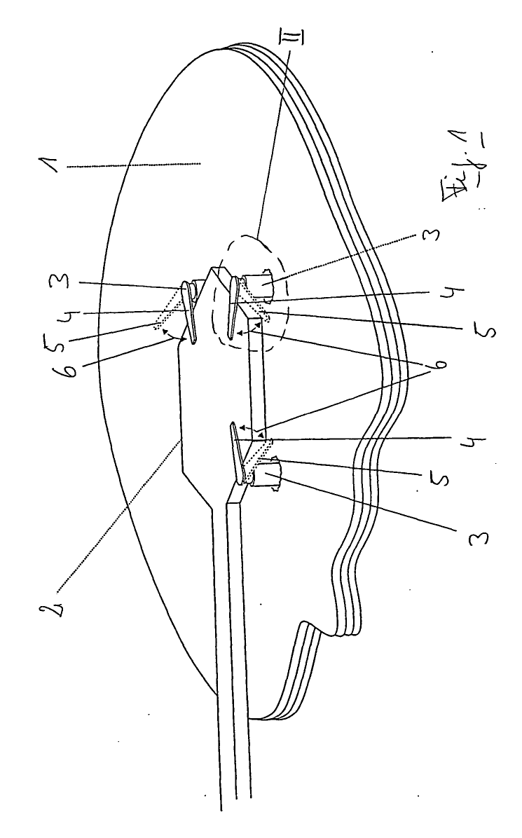

FIG. 1: a tissue extract from the retina with a microcontact foil which lies epiretinal on the tissue section and is attached with swivellable anchoring devices;

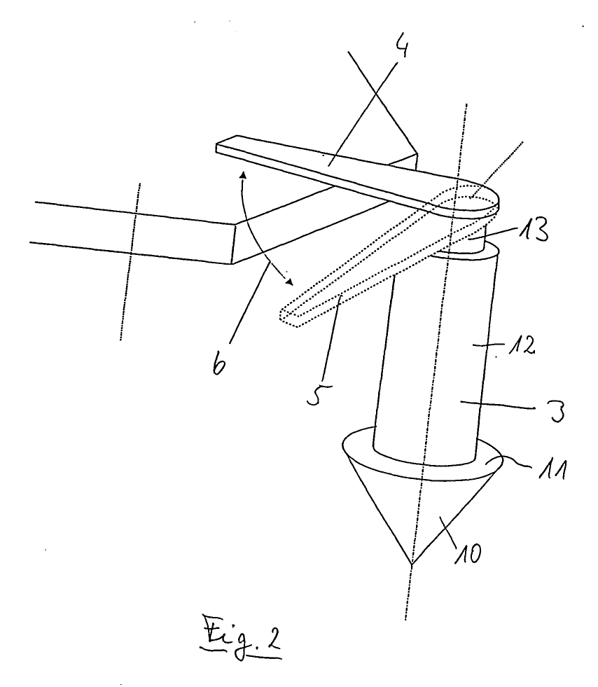

FIG. 2: the anchoring device according to FIG. 1 in an enlarged view; and

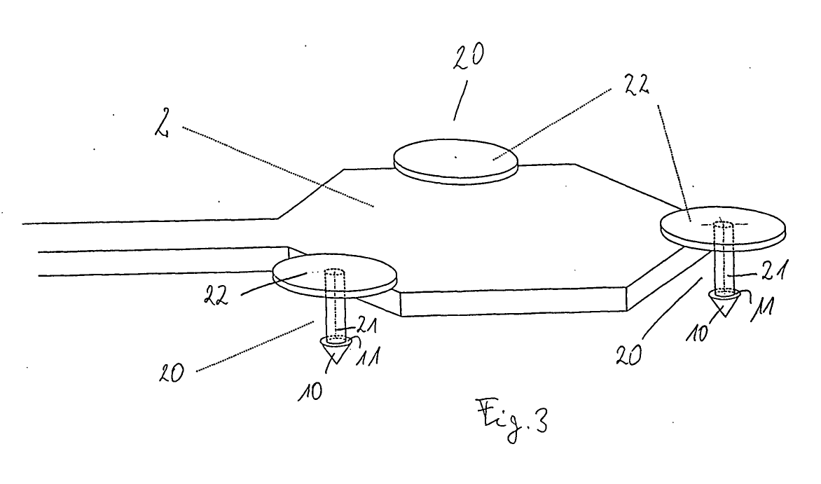

FIG. 3: an implant according to FIG. 1 which is attached with circular disc-like anchors and can be removed from the fixing by bending or pulling.

FIG. 1 shows diagrammatically an extract from a retina 1 which is shown with the side facing the lens at the top. A microcontact foil 2 lies flat on the surface of the retina 1. The non-visible contacts of the microcontact foil 2 are facing the retina 1 in order there to be able to generate sensory perception in the known manner by electrical impulses.

The microcontact foil 2 in this view is secured by three anchoring devices 3, each of which has a swivellable lever 4 as a fixing head. The levers 4 are shown in their anchoring position. They can be swivelled into a release position 5 about an axis oriented vertical to the implant 2, which is indicated by the double arrow 6. Section II is shown in more detail in FIG. 2.

FIG. 2 shows the anchoring device 3 in an enlarged view, where for clarification the retina 1 is not shown. The anchoring head 3, in the same way as micro tacks, has for penetrating the retina a conical tip 10 which on the side facing away from the tip has a flat surface 11 as an undercut. Due to this design, the anchoring device 3, when inserted through the retina, pigmented epithelium and choroid, is fastened so that it cannot be removed again without damaging the tissue. From the flat surface 11, a cylindrical shank 12 continues in the direction of the retina surface visible in FIG. 1. The shank 12 is hollow and surrounds a shaft 13 mounted therein which is connected with the lever. The rotatability of the shaft 13 allows the swivelling of the lever from the anchoring position 4 into the release position 5 in the direction of double arrow 6.

To implant the microcontact foil 2, this can either be inserted as with the known micro tacks and then immediately attached to the anchoring device 3, where the anchoring devices 3 as shown in FIG. 1 are inserted next to the microcontact foil and the lever is in the anchoring position 4. Alternatively, first the anchoring devices 4 can be inserted at the required point, where the levers are then in the release position 5. Then the microcontact foil 2 can be inserted between the anchoring devices 3 and the levers swivelled into the anchoring position 4. In both cases, the implant is secured at the desired implant site.

If the implant is faulty or must be replaced with another implant, the levers can be swivelled into the release position 5, the implant can then be removed without damaging the tissue and the new implant inserted. This is advantageous in particular as the tissue in contact with the implant must in any case bear a slight mechanical strain. Damage to the tissue, as is unavoidable with conventional micro tacks, is excluded.

FIG. 3 shows another embodiment of the present invention in which the microcontact foil 2 is attached with anchoring devices 20 which, in the same way as the anchoring devices 3 in FIGS. 1 and 2, are inserted in the retina directly next to the microcontact foil 2. The fixing devices 20 have a shank 21 which is attached to the flat surface 11 and carries a relatively large circular disc-like anchoring head 22. The anchoring head 22 overlaps with the microcontact foil 2 and thus secures this at the implantation site.

The microcontact foil 2 can be attached also in two ways in this embodiment. In the first variant, the microcontact foil 2 is brought to the proposed implantation site and then attached as with the known micro tacks, where the anchoring devices 20 are also inserted in the retina directly next to the implant. The implant is then held by the large circular disc-like anchoring heads 22.

In another variant, first the implantation site can be defined by the attachment of anchoring devices 20 and then the microcontact foil 2 inserted by bending or sliding beneath the anchoring heads 22.

To remove the microcontact foil 2, this can be bent or folded, or depending on the embodiment pulled sideways beneath the anchoring heads 22. A new microcontact foil can then be implanted correspondingly. The anchoring devices 20 remain in the tissue and assume the anchoring function for the new microcontact foil.

Other embodiments of the present invention not shown are also possible. Thus, for example, the levers in the anchoring device shown in FIGS. 1 and 2 can be swivelled not parallel to the surface of the implant but vertical. It is also possible not to provide a bearing for shaft 13 in the shank 12 but to allow a rotation of the anchoring device as a whole by swivelling the lever from the anchoring position 4 into the release position 5. In each case, the removal of an implanted microcontact foil causes essentially less tissue damage with the anchoring devices according to the invention. It is therefore to the benefit of the implant wearer that an exchange of implant can be made more readily than with the known fixing devices.

The present invention thus creates a removable fixing or holding of microcontact foils, in particular to retina tissue. To this end, the micro tacks are positioned either at the edge of the contact foil without passing through a hole in the foil so that for re-explantation the very flexible foil can be extracted by bending beneath the anchoring heads of the micro tacks which overlap the foil in sections (see FIG. 3); alternatively, the anchoring heads are mobile but attached by their own fixing to the actual anchoring structure (or the structure of the head protruding on one side is firmly connected with the anchoring structure and is rotated as a whole in the retina) and can be rotated for re-explantation (see FIG. 2) or otherwise extracted without essentially changing the position of the fixing structure penetrating the retina.

1-3. (canceled)

4. A removable fixing device for implantable microcontact foils as neuroimplants, comprising:
an anchoring head that extends beyond an inplant on a side thereof that faces away from tissue that is to receive said implant for a fixing purpose, wherein such fixing can be released for a re-explantation by a pulling of a microcontact foil out from beneath said anchoring head.

5. A removable fixing device for implantable microcontact foils as neuroimplants, comprising:
an anchoring head that extends beyond an inplant surface for a fixing purpose, wherein for releasing such fixing, said anchoring head can be rotated, swiveled, folded or pulled.

6. A removable fixing head for implantable microcontact foils as neuroimplants, comprising:
an anchoring head, and
anchoring structure protruding through tissue that is to receive an implant, wherein said anchoring head is removably connectable with said anchoring structure.
